(19) United States
(12) Patent Application Publication
(10) Pub. No.: US 2023/0347142 A1
(43) Pub. Date: Nov. 2, 2023

(54) PACING LEAD CONVERSION TOOL

(71) Applicant: Pacesetter, Inc., Sylmar, CA (US)

(72) Inventors: Steve Chantasirivisal, Los Angeles, CA (US); Keith Victorine, Santa Clarita, CA (US); Wesley Alleman, Santa Clarita, CA (US)

(21) Appl. No.: 18/100,532

(22) Filed: Jan. 23, 2023

Related U.S. Application Data
(60) Provisional application No. 63/335,423, filed on Apr. 27, 2022.

(51) Int. Cl. A61N 1/05

(52) U.S. Cl. CPC A61N 1/057; A61N 2001/058; A61N 1/362

(57) ABSTRACT

A pacing lead conversion tool and a method of using the pacing lead conversion tool to reversibly convert an extendable cardiac pacing lead into a fixed cardiac pacing lead. The pacing lead conversion tool includes a locking lumen configured to receive a pacing lead. The locking lumen includes a connector body segment to receive a connector body of the pacing lead in a first friction fit and a pin segment to receive a connector pin of the pacing lead in a second friction fit. The friction fits allow for relative rotation between a lead body of the pacing lead and the connector pin when the pacing lead conversion tool is twisted, and prevents relative rotation between the lead body and the connector pin when the cardiac pacing lead is burrowed through a target tissue. Other embodiments are also described and claimed.

This application claims the benefit of priority of U.S. Provisional Patent Application No. 63/335,423, filed Apr. 27, 2022, entitled “Pacing Lead Conversion Tool,” and that patent application is incorporated herein by reference in its entirety to provide continuity of disclosure.

### BACKGROUND

### Field

Embodiments related to leads for conducting electrical signals to and from target tissue are disclosed. More particularly, embodiments related to accessories used with pacing leads for delivering electrical charges to cardiac tissue are disclosed.

### Background Information

Fixed helix leads have become the standard of care for pacing applications such as physiologic pacing. With respect to physiologic pacing, for example, such leads are delivered through fixed or deflectable catheters to specific targets in the His Bundle or the Left Bundle Branch (LBB).

Extendable/retractable cardiac pacing leads can also be used in physiological pacing applications. Such leads commonly include a stylet lumen, which allows for a stylet to be placed through the lead for support within the target anatomy. Such leads also include a connector pin, which is controlled relative to a lead body during the implant procedure. More particularly, the connector pin can be rotated to extend or retract a helix for active fixation at the target tissue. For example, a tool can be clipped onto the connector pin and a physician can hold the lead body in one hand while rotating the tool with another hand to cause rotation of the connector pin. Accordingly, the helix can be protected within the lead body during delivery to the target site and, after delivery to the site, can be extended to engage the target tissue. A physician can then push the lead through the target tissue to burrow the lead toward a target pacing site.

### SUMMARY

In an embodiment, a pacing lead conversion tool includes a tool body. The tool body includes a body wall extending around a central lumen. The central lumen extends along a longitudinal axis from a distal tool end to a proximal tool end. The central lumen includes a locking lumen segment having a connector body subsegment and a pin subsegment. The connector body subsegment is sized to receive a connector body of a pacing lead in a first friction fit. The pin subsegment is sized to receive a connector pin of the pacing lead in a second friction fit. The second friction fit is tighter than the first friction fit.

In an embodiment, a pacing lead conversion tool includes a tool body including a body wall extending around a central lumen. The central lumen extends along a longitudinal axis from a distal tool end to a proximal tool end. The central lumen includes a locking lumen segment and a funnel segment. The locking lumen segment includes a connector body subsegment and a pin subsegment. The funnel segment tapers from a proximal opening at the proximal tool end to a distal opening adjacent to the pin subsegment. The distal opening has a smaller diameter than the pin subsegment.

In an embodiment, a method includes inserting a pacing lead into a pacing lead conversion tool. A connector body of the pacing lead is received in a central lumen of the pacing lead conversion tool in a first friction fit. A connector pin of the pacing lead is received within the central lumen of the pacing lead conversion tool in a second friction fit. The second friction fit is tighter than the first friction fit. The method includes torquing the pacing lead conversion tool such that the connector pin and the pacing lead conversion tool rotate relative to the connector body.

The above summary does not include an exhaustive list of all embodiments of the present invention. It is contemplated that the invention includes all systems and methods that can be practiced from all suitable combinations of the various embodiments summarized above, as well as those disclosed in the Detailed Description below and particularly pointed out in the claims filed with the application. Such combinations have particular advantages not specifically recited in the above summary.

### BRIEF DESCRIPTION OF THE DRAWINGS

FIG. 1 is a perspective view of a pacing lead conversion tool, in accordance with an embodiment.

FIG. 2 is a cross-sectional view of a pacing lead conversion tool mounted on a cardiac pacing lead, in accordance with an embodiment.

FIG. 3 is a cross-sectional, top view of a pacing lead conversion tool, in accordance with an embodiment.

FIG. 4 is a cross-sectional, side view of a pacing lead conversion tool, in accordance with an embodiment.

FIG. 5 is a rear view of a pacing lead conversion tool, in accordance with an embodiment.

FIG. 6 is a perspective view of a pacing lead conversion tool, in accordance with an embodiment.

FIG. 7 is a cross-sectional view of a pacing lead conversion tool mounted on a cardiac pacing lead, in accordance with an embodiment.

FIG. 8 is a cross-sectional, top view of a pacing lead conversion tool, in accordance with an embodiment.

FIG. 9 is a cross-sectional, side view of a pacing lead conversion tool, in accordance with an embodiment.

FIG. 10 is an end view of a pacing lead conversion tool, in accordance with an embodiment.

FIG. 11 is a perspective view of a stylet loaded through a pacing lead conversion tool into a cardiac pacing lead, in accordance with an embodiment.

FIG. 12 is a top view of a pacing lead conversion tool mounted on a cardiac pacing lead, in accordance with an embodiment.

FIG. 13 is a top view of a pacing lead conversion tool mounted on a cardiac pacing lead, in accordance with an embodiment.

FIG. 14 is a pictorial view of a pacing system analyzer connected to a cardiac pacing lead through slots of a pacing lead conversion tool, in accordance with an embodiment.

FIG. 15 is a flowchart of a method of implanting and monitoring a pacing lead, in accordance with an embodiment.

### DETAILED DESCRIPTION

Embodiments describe a pacing lead conversion tool and a method of using the pacing lead conversion tool to reversibly convert an extendable cardiac pacing lead into a fixed cardiac pacing lead. The pacing lead conversion tool may be used during implantation of cardiac pacing leads. For example, the tool can assist in delivering cardiac pacing leads to a Left Bundle Branch (LBB). The pacing lead conversion tool may, however, be used during implantation of pacing leads at other physiological sites, such as within the brain or spine, to name a few alternative applications.

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the embodiments. In other instances, well-known processes and manufacturing techniques have not been described in particular detail in order to not unnecessarily obscure the description. Reference throughout this specification to “one embodiment,” “an embodiment,” or the like, means that a particular feature, structure, configuration, or characteristic described is included in at least one embodiment. Thus, the appearance of the phrase “one embodiment,” “an embodiment,” or the like, in various places throughout this specification are not necessarily referring to the same embodiment. Furthermore, the particular features, structures, configurations, or characteristics may be combined in any suitable manner in one or more embodiments.

The use of relative terms throughout the description may denote a relative position or direction. For example, “distal to” may indicate a first direction away from a reference point. Similarly, “proximal to” may indicate a location in a second direction away from the reference point and opposite to the first direction. Such terms are provided to establish relative frames of reference, however, and are not intended to limit the use or orientation of a pacing lead conversion tool to a specific configuration described in the various embodiments below.

Existing clip-on tools for rotating a connector pin to advance a helix of an extendable cardiac pacing lead do not prevent rotation between a lead body and the connector pin. When the physician manipulates the lead to burrow the lead deep into the physiologic target, back pressure from the tissue and twisting of the lead body can cause the helix to spontaneously retract into the lead body. Such retraction can undermine fixation of the lead and compromise pacing of the target tissue. Accordingly, a tool to rotate the connector pin, which also prevents spontaneous helix retraction during the burrowing process, is needed.

In an aspect, a pacing lead conversion tool can temporarily convert an extendable cardiac pacing lead into a fixed cardiac pacing lead. More particularly, the pacing lead conversion tool can allow a physician to rotate a connector pin to extend a helix of the lead, however, the tool can resist relative rotation between the connector pin (and the helix) and the lead body when the physician is burrowing the lead into target tissue. The pacing lead conversion tool can also facilitate stylet exchange into the cardiac pacing lead, and connection of surgical cables to the cardiac pacing lead for use with a pacing system analyzer (PSA). Accordingly, the pacing lead conversion tool can reduce a likelihood of spontaneous helix retraction and therefore contribute to effective delivery and pacing.

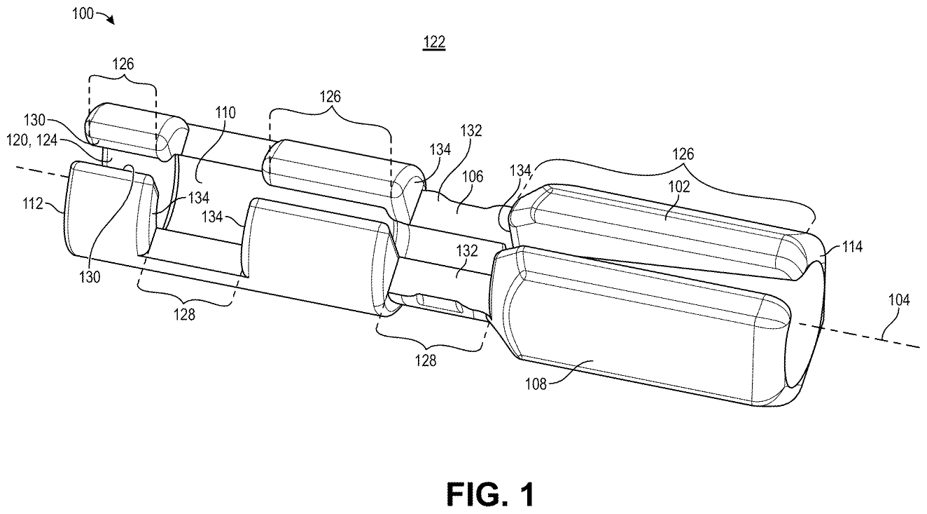

Referring to FIG. 1, a perspective view of a pacing lead conversion tool is shown in accordance with an embodiment. Pacing lead conversion tool 100 can be mounted on an extendable cardiac pacing lead to convert the lead into a fixed helix cardiac pacing lead during implantation. More particularly, the pacing lead conversion tool 100 can constrain a connector pin of the pacing lead relative to a lead body of the pacing lead. The constraint may be torque-limited, however. Application of a torque above a predetermined limit to the pacing lead conversion tool 100, when the pacing lead conversion tool is mounted on the connector pin and the lead body, can cause the pacing lead conversion tool and the connector pin to rotate together relative to the lead body.

In an embodiment, the pacing lead conversion tool 100 includes a tool body 102. The tool body 102 can be monolithic or formed from several components connected to each other. The tool body 102 may include a longitudinal axis 104 extending centrally through the body. A body wall 106 of the tool body 102 can have an outer surface that may be gripped by a physician. For example, the body wall 106 can define a handle 108 that is disposed about the longitudinal axis 104.

The handle 108 can have a gripping surface, e.g., a hexagonal cross-section, ridges, knurling, etc. to facilitate gripping and twisting of the pacing lead conversion tool 100 by the physician. The handle 108 may be symmetrically disposed about the longitudinal axis 104. Alternatively, the handle 108 may have an asymmetric distribution about the longitudinal axis 104. For example, the handle 108 may have a lever extending laterally in a single direction.

In an embodiment, the body wall 106 extends around a central lumen 110 of the pacing lead conversion tool 100. The central lumen 110 can extend along the longitudinal axis 104 through the tool body 102 from a distal tool end 112 to a proximal tool end 114. The distal tool end 112 can be a distalmost, distally-facing surface of the tool body 102. The proximal tool end 114 may be a proximalmost, proximally-facing surface of the handle 108.

As described below, the central lumen 110 can have segments and subsegments adapted to different purposes. For example, a portion of the central lumen 110 may be adapted to receive a proximal end of a cardiac pacing lead. The cardiac pacing lead can be an extendable pacing lead, and the central lumen 110 can be sized such that the tool body 102 grips a connector pin of the pacing lead. Furthermore, a portion of the central lumen 110 may be adapted to allow stylets to be exchanged into the pacing lead. A size and shape of the central lumen 110 can facilitate the tool functions described below.

The pacing lead conversion tool 100 can include a side slot 120 to expose the central lumen 110 to a surrounding environment 122. More particularly, the side slot 120 can extend through the body wall 106 of the tool body 102 from the central lumen 110 to the surrounding environment 122. Accordingly, the side slot 120 provides an opening between the central lumen 110 and the surrounding environment 122. Objects, such as a stylet or an electrical connector, can therefore pass through the side slot 120 into the central lumen 110.

In an embodiment, the side slot 120 can be an intermittent slot 124. More particularly, the side slot 120 can include several slot segments 126 longitudinally separated by several intervening connector openings 128. The slot segments 126 and the connector openings 128 can combine to form a continuous opening from the proximal tool end 114 to the distal tool end 112. The opening, although continuous, may not be consistent. For example, a circumferential width of the segments forming the continuous opening can vary over a length of the pacing lead conversion tool 100.

The circumferential width of the slot segments 126 of the side slot 120 may be less than the width of the connector openings 128. For example, the circumferential width of the slot segments 126, measured laterally between circumferentially-facing slot segment surfaces 130 of the slot segments 126, can be less than a diameter of the central lumen 110 exposed through the slot segment 126. The central lumen 110 exposed through the slot segments 126 can have a diameter of, e.g., 0.135 inch, and the circumferential width of the slot segments 126 can be less than 0.135 inch, e.g., 0.040 inch. By contrast, the circumferential width of the connector openings 128, measured laterally between circumferentially-facing connect slot surfaces 132 of the connector openings 128, can be larger than the circumferential width of the slot segments 126. In an embodiment, the circumferential width of the connector openings 128 is at least as large as the diameter of the central lumen 110. More particularly, the connector openings 128 can extend through the body wall 106 to the longitudinal axis 104, and thus, an entirety of the central lumen 110 can be exposed through the connector opening 128.

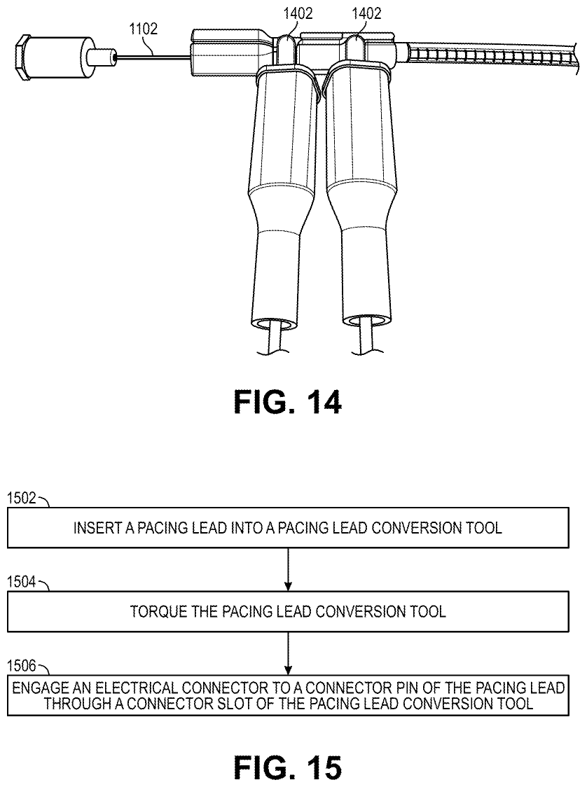

Given that the connector opening 128 can have a larger circumferential width than the slot segments 126, the connector openings 128 can interrupt the slot segments 126. The interrupted slot segments 126 may have slot segment faces 134 that face longitudinally across the connector openings 128. For example, the slot segment faces 134 can face each other through the connector openings 128. The connector openings 128 can therefore be defined between the slot segment faces 134 and the connector opening surfaces 132, and can provide an opening large enough to receive an electrical connector (FIG. 14). Furthermore, the connector openings 128 can interrupt the slot segments 126, which are not large enough to receive the electrical connector, but which can receive a stylet laterally through the body wall 106. The continuous opening between the distal tool end 112 and the proximal tool end 114 accordingly has a varied width that exposes different degrees of the central lumen 110 over the tool length. As now shall be described, just as the side slot 120 can vary in size over the tool length, so may the central lumen 110 vary in size over the tool length.

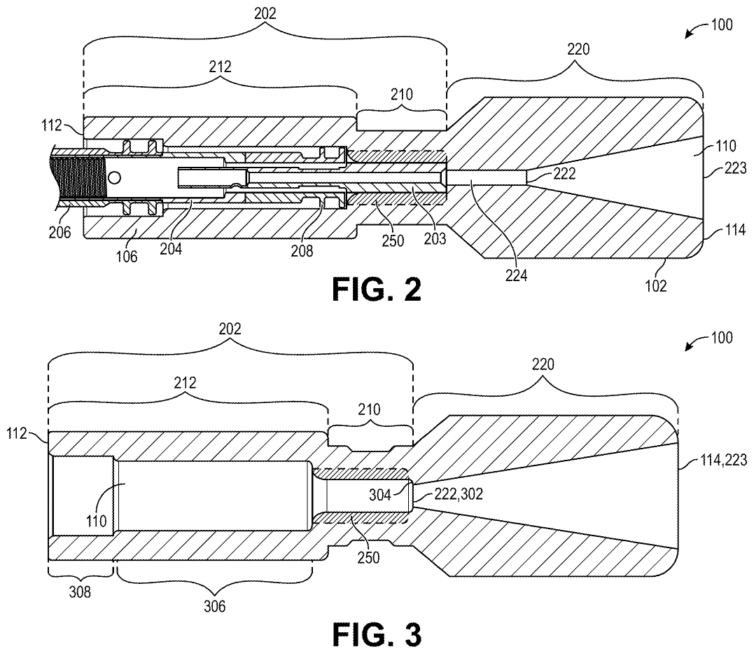

Referring to FIG. 2, a cross-sectional view of a pacing lead conversion tool mounted on a cardiac pacing lead is shown in accordance with an embodiment. The central lumen 110 can be divided into two or more segments between the distal tool end 112 and the proximal tool end 114. The central lumen 110 can include a locking lumen segment 202 extending proximally from the distal tool end 112 through the tool body 102. The locking lumen segment 202 may be sized and configured to receive and engage a proximal end of a pacing lead 204. For example, the pacing lead 204 may be an extendable pacing lead having a lead connector. The lead connector can be an IS-1 or DF4 lead connector. For example, the lead connector can include a connector pin 203, and the connector pin 203 can be an IS-1 or DF4 connector pin 203. Rotation of the connector pin 203 can advance or retract a fixation helix (not shown) of the pacing lead 204 relative to a lead body 206. The central lumen 110 can be sized and shaped such that an inner surface of the body wall 106, which extends around the central lumen 110, can engage one or more of the connector pin 203 or a connector body 208 of the lead body 206.

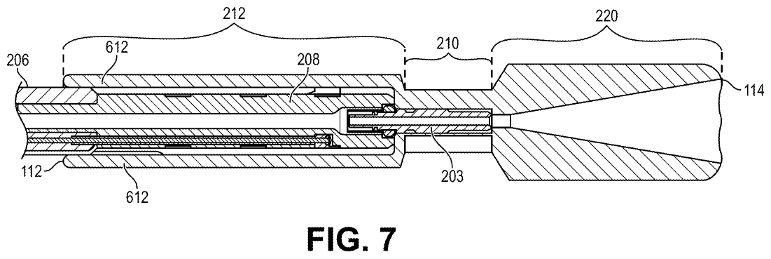

The connector body 208 may include a seal or a seal zone of the pacing lead 204. For example, IS-1 connectors and DF4 connectors may have physical seals and/or seal zones. An IS-1 seal, as shown in FIG. 2, can include sealing rings. The sealing rings can be in each of two sealing-ring zones on the lead connector, and may be intended to bear on seal zones within a biostimulator. A DF4 connector (FIG. 7) can have pristine seal zones, which may be defined as zones on the lead connector that seal with mating seals in a connector cavity of a biostimulator. The DF4 connector may also include a contact zone and a boot zone. It is contemplated that the connector body 208 can include any of such structures, and that the pacing lead conversion tool 100 can grip any of such structures. For example, the tool can be sized to grab the seal/seal zones of an IS-1 connector or a connector boot of a DF4 connector. Similarly, the pacing lead conversion tool 100 can be adapted to fit other lead connector designs to constrain relative motion between several surfaces of the connector according to the principles described herein.

Subsegments of the locking lumen segment 202 can be adapted to receive predetermined portions of the lead connector. The locking lumen segment 202 of the central lumen 110 can have a pin subsegment 210. The pin subsegment 210 can be sized to interact with the connector pin 203 of the pacing lead 204. For example, the pin subsegment 210 can be sized to receive a connector pin 203 of the pacing lead 204 in a friction fit. More particularly, the body wall 106 surrounding and defining the pin subsegment 210 can engage the connector pin 203 in the friction fit when the connector pin 203 is received within the pin subsegment 210. An outer dimension of the pin subsegment 210 can be smaller than an outer dimension of the connector pin 203. Accordingly, the inner surface of the body wall 106 around the pin subsegment 210 can have an interference fit with the connector pin 203. In such case, when the proximal end of the pacing lead 204 is inserted into the central lumen 110, the tool body 102 can receive and engage the connector pin 203 in the friction fit, e.g., a press fit, that effectively binds the tool body 102 to the connector pin 203 and prevents sliding of the connector pin 203 within the central lumen 110 when the tool body 102 is rotated. The tool body 102 can effectively grab the connector pin 203. Accordingly, when the physician twists the tool body 102 while stabilizing the lead body 206, torque is transmitted to the connector pin 203 to rotate the helix of the pacing lead 204.

In an embodiment, the pacing lead conversion tool 100 includes a pin sleeve 250 within the pin subsegment 210. The pin sleeve 250 is shown by cross-hatching in FIG. 2, indicating that the sleeve can fill a portion of the tool body 102 radially inward of the body wall 106. For example, the pin sleeve 250 can be a tubular sleeve loaded into a recess of the tool body 102.

The pin sleeve 250 can be softer than the body wall 106. For example, the pin sleeve 250 can be formed from a soft, pliable material, and the body wall 106 may be formed from a rigid material. By way of example, the pin sleeve 250 can include a tubular sleeve formed from silicone, and the body wall 106 may be molded from acrylonitrile butadiene styrene (ABS), or another polymer that is harder than the silicone used to form the pin sleeve 250.

The soft, pliable inner surface of the tubular sleeve can compress during insertion of the connector pin 203. For example, the inner surface of the tubular sleeve can have a smaller diameter than an outer diameter of the connector pin 203, and thus, the tubular sleeve can be compressed by, and squeeze onto, the connector pin 203. The press fit between the soft pin sleeve 250 and the hard connector pin 203 can generate high friction to resist relative rotation between the pin sleeve 250 and the connector pin 203. More particularly, the pin sleeve 250 and the connector pin 203 can become locked to each other when the pacing lead 204 is inserted into the pacing lead conversion tool 100.

The compliance of the pin sleeve 250 can also allow the pacing lead conversion tool 100 to adjust to variations in connector pin sizes and/or dimensional tolerances. More particularly, an inner dimension of the pin sleeve 250 can be no larger than a minimum dimensional tolerance of the connector pin 203, ensuring that the pin sleeve will receive and grip the connector pin to lock the pacing lead conversion tool 100 to the connector pin 203.

The locking lumen segment 202 of the central lumen 110 can have a connector body subsegment 212. The connector body subsegment 212 can have a larger diameter than the pin subsegment 210. The connector pin 203, which has a smaller outer dimension then the connector body 208, e.g., a seal of an IS-1 connector, may therefore pass through the connector body subsegment 212 to engage the pin subsegment 210.

The connector body subsegment 212 of the locking lumen segment 202 can be sized to interact with the seal of the pacing lead 204. More particularly, the connector body subsegment 212 can be sized to receive the connector body 208 of the pacing lead 204 in a friction fit. More particularly, the body wall 106 surrounding and defining the connector body subsegment 212 can engage the connector body 208 in the friction fit when the connector body 208 is received within the connector body subsegment 212. An outer dimension of the connector body subsegment 212 can be slightly smaller or larger than an outer dimension of the seal. Accordingly, the inner surface of the body wall 106 around the connector body subsegment 212 can interfere with the seal. In such case, when the proximal end of the pacing lead 204 is inserted into the central lumen 110, the tool body 102 can receive and engage the connector body 208 in the friction fit, e.g., a slip fit, that resists but does not entirely prevent sliding of the connector body within the central lumen 110 when the tool body 102 is rotated. The tool body 102 can lightly touch the seal. The friction fit between the body wall 106 surrounding the pin subsegment 210 of the tool and the connector pin 203 may be tighter, however, than the friction fit between the body wall 106 surrounding the connector body subsegment 212 of the tool and the connector body 208. Accordingly, when the physician twists the tool body 102 while stabilizing the lead body 206, the tool body 102 can slide over the seal to allow relative rotation between the connector pin 203 and the lead body 206. In contrast, when less torque is applied to the junction between the tool body 102 and the seal, e.g., when incidental torque is transmitted through the lead body 206 while the physician advances the pacing lead 204 through a cardiac septum, the friction fit can reduce the likelihood of rotation between the seal and the tool body 102, and thus, can reduce the likelihood of spontaneous helix retraction. Accordingly, the pacing lead conversion tool 100 can convert, at least temporarily, an extendable pacing lead 204 into a fixed pacing lead 204.

It will be appreciated from the above description that, in an embodiment, the friction fit between the tool body 102 and the connector body 208 of the pacing lead 204 may be less tight than the friction fit between the tool body 102 and the connector pin 203 of the pacing lead 204. More particularly, the tool body 102 can apply more pressure to the connector pin 203 than the connector body 208. The friction fits can determine the torque that can be applied to the lead components through the tool body 102 before slippage occurs. The tool body 102 may therefore be bound more tightly to the connector pin 203 than the connector body 208, allowing the physician to rotate the connector pin 203 through the tool body 102 while stabilizing the pacing lead 204, which rotates relative to the twisting tool body 102.

A physician may want to exchange stylets during the implantation procedure. For example, more support may be required to advance the pacing lead 204, and the physician may retract a first, more flexible stylet from the lumen of the pacing lead 204 and insert a second, more rigid stylet into the lumen during an exchange procedure. An outer dimension of the stylet may be small, and inserting a distal end of the stylet into a lumen of the connector pin may be challenging, especially in a low-light surgical room setting.

The pacing lead conversion tool 100 can include a funnel to aid in stylet exchange, and more particularly, in insertion of a stylet into the lumen of the pacing lead 204. The funnel may be defined in part by the central lumen 110. More particularly, the central lumen 110 can include a funnel lumen segment 220 having a size and shape that defines the funnel within the tool body 102. The funnel lumen segment 220 may be proximal to the locking lumen segment 202. An inner surface of the tool body 102 around the funnel lumen segment 220 can taper inward from the proximal tool end 114 to a distal funnel end 222. The inner surface can taper smoothly and continuously such that a distal end of the stylet, when inserted into a proximal opening 223 of the funnel lumen segment 220, can slide along the inner surface into a funnel neck 224 of the funnel. The funnel neck 224 can be a lumen having a dimension of a same or similar size to the lumen of the connector pin 203. The stylet may be inserted into the proximal opening 223 and then advanced distally through the funnel neck 224 into the connector pin 203. Accordingly, the pacing lead conversion tool 100 can aid in exchange of stylets during the implantation procedure.

Referring to FIG. 3, a cross-sectional, top view of a pacing lead conversion tool is shown in accordance with an embodiment. As described above, the central lumen 110 can have the locking lumen segment 202, which can include the connector body subsegment 212 and the pin subsegment 210, and the funnel segment 220. In an embodiment, the funnel segment 220 tapers from the proximal opening 223 at the proximal tool end 114 to a distal opening 302 at the distal funnel end 222. The distal opening 302 can be adjacent to the pin subsegment 210. For example, the distal opening 302 of the funnel may be a proximal opening of the pin subsegment 210. More particularly, the funnel may open directly into the pin subsegment 210. Alternatively, as shown in FIG. 2, a transitional lumen, such as the funnel neck 224, can extend from the distal opening 302 to the pin subsegment 210. The transitional lumen can have a constant diameter. In either case, whether the distal opening 302 joins the pin subsegment 210 directly or via a transitional lumen, the distal opening 302 can have a smaller diameter than the pin subsegment 210.

The smaller diameter of the distal opening 302, relative to the pin subsegment 210, can prevent the connector pin 203 from passing entirely through the pacing lead conversion tool 100. More particularly, the body wall 106 forming the distal opening 302 can act as a stop 304, restricting movement of the connector pin 203 when it is engaged with the tool within the pin subsegment 210. The stop 304 can include a ledge jutting radially inward from the body wall 106 surrounding the pin subsegment 210. The stop 304 can locate the connector pin 203 properly relative to the pin subsegment 210 and/or pin sleeve 250. Furthermore, having a smaller distal opening 302 can provide a smooth lead in of a stylet passing distally through the funnel into a lumen of the connector pin 203.

The proximal opening 223 of the funnel subsegment, in contrast to the distal opening 302, has a larger diameter than the connector body subsegment 212 and the pin subsegment 210. The proximal opening 223 is also larger than the distal opening 302. The large proximal opening 223 provides an easily identifiable target that can be quickly engaged with a stylet, even under dim lighting. The funnel can taper gradually from the proximal opening 223 to the distal opening 302. For example, the funnel can include a conical profile having a cone angle in a range of 15° to 20°, e.g., 18°.

In an embodiment, the connector body subsegment 212 can be further divided into a proximal connector body subsegment 306 and a distal connector body subsegment 308. The further subsegments of the connector body subsegment 212 can be sized to receive different portions of the lead connector. For example, the proximal connector body subsegment 306 may be sized to receive a proximal seal of a lead connector and the distal connector body subsegment 308 may be sized to receive a distal seal of the lead connector (FIG. 2). The subsegments may be sized, accordingly, to engage the respective seals with appropriate fits. For example, the proximal connector body subsegment 306 may have a smaller diameter than the distal connector body subsegment 308 to engage the proximal seal of the pacing lead 204 that is smaller than the distal seal of the pacing lead 204. The lengths of the connector body subsegment portions may also be selected to provide appropriate landings for the seals.

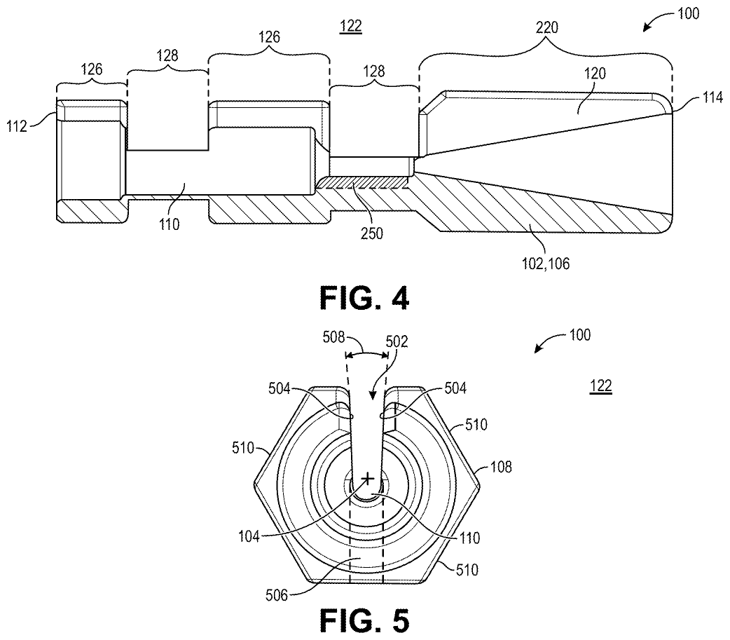

Referring to FIG. 4, a cross-sectional, side view of a pacing lead conversion tool is shown in accordance with an embodiment. In cross-section, the side slot 120 extending through an upper portion of the tool body 102 can be readily contrasted with the continuous body wall 106 extending along the lower portion of the tool body 102. More particularly, the side slot 120 can extend through the body wall 106 of the tool body 102 from the central lumen 110 to the surrounding environment 122, and from the distal tool end 112 to the proximal tool end 114. The side slot 120 can therefore extend through the body wall 106 around the slot segments 128, the connector openings 128, and the funnel segment 220 of the central lumen 110. Accordingly, the side slot 120 can provide a continuous opening over a length of the tool body 102 between the central lumen 110 and the surrounding environment 122. In contrast, the body wall 106 extending along the lower portion of the tool body 102 may be entirely closed. More particularly, a section of the tool body 102 diametrically opposite to the side slot 120 may be entirely solid, having no holes, slots, or openings between the central lumen 110 and the surrounding environment 122. The body wall 106 below the central lumen 110 may therefore fully support the lead connector when it is inserted into the pacing lead conversion tool 100, and separate the lead connector from the surrounding environment 122.

Referring to FIG. 5, an end view of a pacing lead conversion tool is shown in accordance with an embodiment. As described above, the slot segments 126 and the connector openings 128 can combine to form a continuous opening 502 to connect the central lumen 110 to the surrounding environment 122. The continuous opening 502 can be defined between opposing surfaces 504 of the body wall 106 forming the side slot 120. The continuous opening 502 can be diametrically opposite to, e.g., on an opposite side of the longitudinal axis 104, from a solid section 506 of the body wall 106, represented between vertical dashed lines having a width equal to a diameter of the central lumen 110. The solid section can therefore appose any object, e.g., a stylet, an electrical connector, or a lead connector, that is inserted into the central lumen 110.

In an embodiment, the opposing surfaces 504 defining the continuous opening 502 in the cross-section may be angled relative to each other, rather than parallel. For example, planes extending through respective surfaces of the opposing surfaces 504 can be offset from each other by an opening angle 508. The opening angle 508 may be in a range of 2° to 10°, e.g., 4°. The opening angle 508 provides for a lead-in that receives a stylet and guides it into the central lumen 110. The opening angle 508 therefore creates a tapered continuous opening 502 that the stylet can be more easily loaded into.

The handle 108 can have a grip surface 510 facing radially outward from the longitudinal axis 104. The grip surface 510 may have a polygonal profile. For example, the profile may be hexagonal, pentagonal, etc. The polygonal profile can facilitate gripping the handle 108. The handle 108 may include additional features, such as a knurled surface, to facilitate effective gripping.

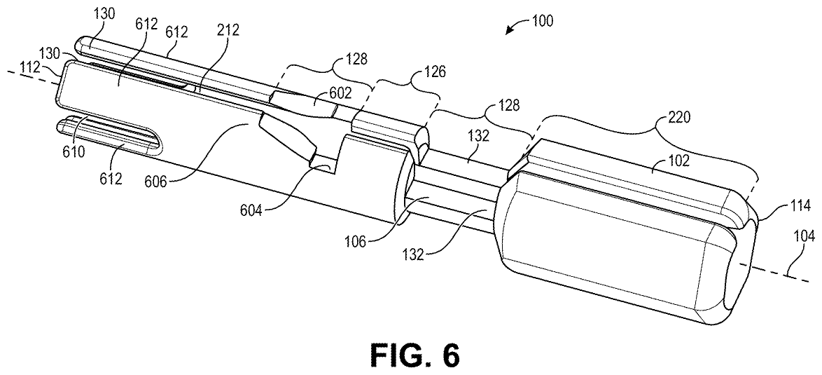

Referring to FIG. 6, a perspective view of a pacing lead conversion tool is shown in accordance with an embodiment. The pacing lead conversion tool 100 shown in FIG. 6 may be configured to receiving a DF4 lead connector. Several of the features of the pacing lead conversion tool 100 are similar or identical to a pacing lead conversion tool 100 adapted to receive an IS-1 lead connector. Such features have corresponding numerals in the pacing lead conversion tool 100 configured to receive the DF4 lead connector and their descriptions shall not be repeated in the interest of brevity. The pacing lead conversion tool 100 embodiment shown in FIG. 6 may include several features that differ from the embodiment shown in FIG. 1, however, and those features are described below.

In an embodiment, one or more of the connector openings 128 incudes a guide surface 602. The guide surface 602 can be a tapering surface that acts to guide an electrical connector, such as an alligator clip, toward a clip surface 604. For example, when the alligator clip is clipped onto the guide surface 602, it can slide down the sloped surface toward the clip surface 604. The alligator clip may then clip onto the clip surface 604 and/or clip onto the connector body 208 located within the central lumen 110 adjacent to the clip surface 604. Accordingly, the guide surface 602 can taper radially inward from an outer surface 606 of the body wall 106 to the clip surface 604 to guide the electrical connector radially inward toward the lead connector surface.

The pacing lead conversion tool 100 may include a body wall 106 that adjusts to a diameter of a pacing lead 204. In an embodiment, the pacing lead conversion tool 100 includes several relief slots 610 between cantilever portions 612 of the body wall 106. The relief slots 610 separate the body wall portions and allow the cantilever portions 612 to flex outward. More particularly, the relief slots 610 can extend longitudinally from the distal tool end 112 through the body wall 106 radially outward of the connector body subsegment 212 to a slot depth. The slot depth defines the length of the flexible, cantilever portions 612. The longer the cantilevers, the more flexible the cantilevers are.

Referring to FIG. 7, a cross-sectional view of a pacing lead conversion tool mounted on a cardiac pacing lead is shown in accordance with an embodiment. When the pacing lead 204 is inserted into the central lumen 110 of the pacing lead conversion tool 100, the flexible wall portions can adjust to a dimension of the lead connector. For example, the cantilever portions 612 can flex outward around the lead body 206. The flexed cantilever portions 612 can contact and grip the lead body 206, and thus, can grip the lead body. The gripped portion can include the connector body 208, e.g., a seal zone, or another portion of the pacing lead 204 that is rotatable relative to the connector pin 203. Accordingly, when the connector body subsegment 212 of the central lumen 110 receives the lead connector, the body wall 106 surrounding the connector body subsegment 212, e.g., the cantilever portions 612, can grip and/or constrain the connector body 208.

As described above, the connector pin 203 can be inserted into the pin subsegment 210 of the central lumen 110 and the body wall 106 surrounding the pin subsegment 210 can form a friction fit against the connector pin 203. The friction fit with the connector pin 203 can be tighter than the friction fit between the cantilever portions 612 and the lead body 206. Accordingly, although the pacing lead conversion tool 100 can constrain relative rotation between the lead body 206 and the connector pin 203, the components may rotate relative to each other when a predetermined torque is applied to the tool while a user holds the lead body 206. More particularly, the predetermined torque can rotate the tool in unison with the connector pin 203, and the connector pin can rotate relative to the lead body 206.

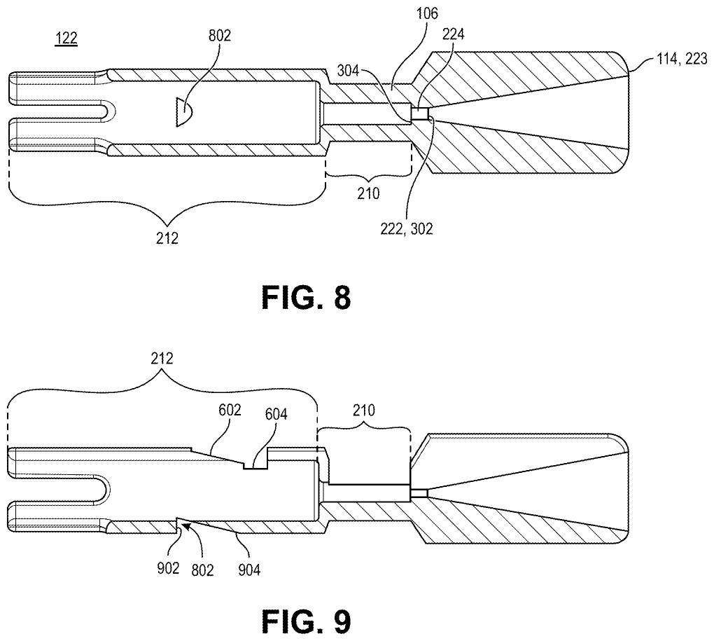

Referring to FIG. 8, a cross-sectional, top view of a pacing lead conversion tool is shown in accordance with an embodiment. In cross-section, it is evident that the pacing lead conversion tool 100 configured to receive a DF4 lead connector has features similar to those present in the pacing lead conversion tool 100 configured to receive an IS-1 lead connector. Again, features of the embodiments are interchangeable and may be combined in different configurations within the scope of this description. Such features include the stop 304, the funnel neck 224, etc.

In an embodiment, the pacing lead conversion tool 100 includes a clip notch 802 on an opposite side of the body wall 106 from the side slot 120. The clip notch 802 can be located diametrically opposite to the connector opening 128 having the tapered guide surface 602. The clip notch 802 can include a recessed notch extending into the outer surface 606 of the body wall 106. In an embodiment, a portion of the clip notch 802 extends fully through the body wall 106, forming a hole extending from the central lumen 110 to the surrounding environment 122. Accordingly, the pacing lead conversion tool 100 can include an opening along the otherwise solid section 506.

Referring to FIG. 9, a cross-sectional, side view of a pacing lead conversion tool is shown in accordance with an embodiment. In profile, the clip notch 802 can have a transverse wall 902 and a tapered wall 904. The tapered wall 904 can taper distally from the outer surface 606 of the body wall 106 toward the transverse wall 902. The tapered wall 904 can, like the guide surface 602, bias a clip of an electrical connector radially inward to rest within the clip notch 802. When the clip notch 802 extends fully through the body wall 106 to form the hole, the electrical connector may contact a lead connector located within the connector body subsegment 212.

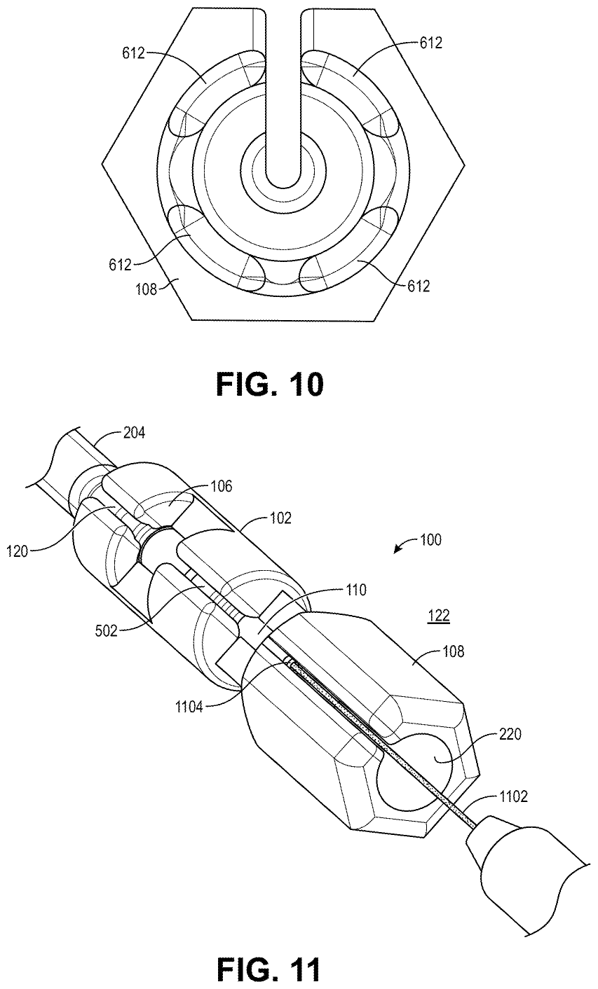

Referring to FIG. 10, an end view of a pacing lead conversion tool is shown in accordance with an embodiment. The end view, directed along the longitudinal axis 104 in a proximal direction, reveals an end profile of the cantilever portions 612. The profiles can be arc-shaped segments, divided from a tubular cross-section. The profiles of the cantilevered portions can have a same width and may be symmetrically disposed about the longitudinal axis 104. In alternative embodiments, the profiles of the cantilevered portions may have varied lengths and/or may be asymmetric about the longitudinal axis 104.

The pacing lead conversion tool 100 may be used with or without a stylet. Stylets provide support to the pacing lead 204 during advancement to a target implantation site, e.g., such as high on a septum of a right ventricle, and while burrowing through the target implantation site to the target pacing site, e.g., a LBB.

Referring to FIG. 11, a side view of a stylet loaded through a pacing lead conversion tool into a cardiac pacing lead is shown in accordance with an embodiment. In an embodiment, the pacing lead conversion tool 100 can be loaded over a stylet that is inserted into the pacing lead 204. The pacing lead conversion tool 100 can include a side slot 120 extending through the body wall 106 of the tool body 102 from the central lumen 110 to a surrounding environment 122. The side slot 120 can extend longitudinally from the distal tool end 112 to the proximal tool end 114. A width of the side slot 120 may be larger than an outer dimension of a stylet.

A stylet 1102 may be inserted into a lead lumen 1104 of the lead body 206. When the stylet 1102 is in place within the lead lumen 1104, the pacing lead conversion tool 100 can be inserted over the stylet 1102 by passing the stylet 1102 laterally through the side slot 120. The side slot 120 can be a continuous opening 502 over the length of the lead conversion tool 100. The stylet 1102 may therefore pass through the opening to be received within the central lumen 110. When a stylet 1102 is aligned with the central lumen 110 of the tool body 102, the tool body 102 can be advanced to engage the proximal end of the pacing lead 204. Accordingly, the pacing lead conversion tool 100 can be placed on the pacing lead 204 without removing the stylet 1102.

In an embodiment, the stylet 1102 can be inserted and/or removed from the lead lumen 1104 when the pacing lead 204 is in place within the central lumen 110 of the pacing lead conversion tool 100. For example, the stylet 1102 can be retracted and removed from the lead lumen 1104. Furthermore, a distal tip of the stylet 1102 can be advanced distally through the funnel of the handle 108. The funnel can guide the distal tip toward the distal opening 302 of the funnel subsegment, to align the distal tip with the lead lumen 1104. Advancing the stylet 1102 further will insert the distal tip into the lead lumen 1104. The stylet 1102 can be pushed distally to drive the distal tip through the lead lumen 1104 to provide support to the lead body 206.

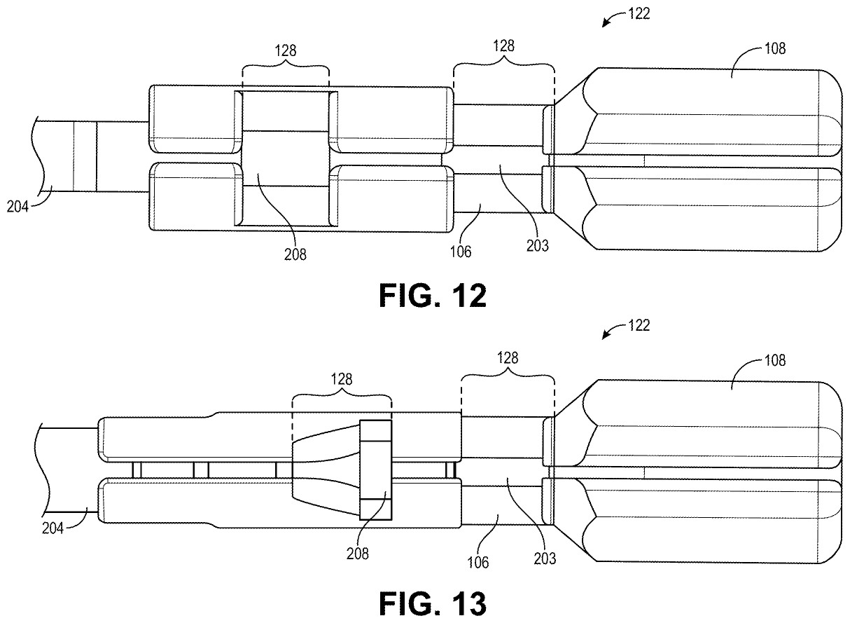

Referring to FIG. 12, a top view of a pacing lead conversion tool mounted on a cardiac pacing lead is shown in accordance with an embodiment. The pacing lead conversion tool 100 can facilitate electrical contact with the connector pin 203 and/or connector body 208 of the pacing lead 204. More particularly, electrical connectors of a PSA may be attached to the lead while the conversion tool is in place. By not requiring removal of the pacing conversion tool, the tool can continue to lock the pacing lead 204 in a fixed lead configuration that reduces the likelihood of spontaneous helix retraction.

In an embodiment, one or more connector openings 128 extend through the body wall 106 of the tool body 102. More particularly, the connector opening(s) 128 extend through the body wall 106 from the locking lumen to the surrounding environment 122. The slots expose the central lumen 110 and, more particularly, the pacing lead 204 within the central lumen 110 to the surrounding environment 122. For example, PSA cable connection points on an IS-1 lead connector can be exposed through the slots. The PSA cable connection points can be points on the connector pin 203 and/or the connector body 208 of the lead connector.

Referring to FIG. 13, a top view of a pacing lead conversion tool mounted on a cardiac pacing lead is shown in accordance with an embodiment. The connector openings 128 can extend through the body wall 106 of the tool body 102 to align with corresponding PSA cable connection points on a DF4 lead connector. More particularly, the connector opening(s) 128 can extend through the body wall 106 from the locking lumen to the surrounding environment 122, and can expose the connector pin 203 and/or the connector body 208 of the lead connector when the pacing lead 204 is received within the conversion tool.

Referring to FIG. 14, a pictorial view of a pacing system analyzer connected to a cardiac pacing lead through slots of a pacing lead conversion tool is shown in accordance with an embodiment. A longitudinal width of the connector opening 128 may be large enough to allow an electrical connector 1402, e.g., an alligator clip, to access the pacing lead 204 through the connector opening 128. For example, the electrical connector 1402 can be clipped onto the pacing lead 204 through the connector opening 128. Surgical cables connected to the electrical connector 1402 can transmit electrical signals between the pacing lead 204 and the PSA. The PSA can measure electrical values through the surgical cables, to determine whether the pacing lead 204 is properly located. For example, the PSA can detect whether the distal end of the pacing lead 204 is at the LBB.

Referring to FIG. 15, a flowchart of a method of using a pacing lead conversion tool to implant a pacing lead is shown in accordance with an embodiment. The operations of the method may be performed in any order, and the description below is not limiting of the operational sequence. Conversion of an extendable pacing lead into a fixed pacing lead may be particularly useful in LBB applications. Accessing the LBB can require the pacing lead 204 to be advanced through one or more centimeters of tissue, and thus, substantial back pressure and incidental forces may be applied to the helix during device burrowing that can cause the helix to spontaneously retract. Use of the pacing conversion tool as described herein can, however, fix the helix in an extended state during deep septal implant.

A workflow for achieving deep septal implantation of the pacing lead 204 at a LBB can include advancing a delivery catheter to an implantation target high on a septum of a right ventricle. The pacing lead 204 can be advanced through the delivery catheter to the target implantation site with or without a stylet 1102. During advancement through the catheter, helix may be retracted within a distal header of the pacing lead 204 to remain protected as it traverses the catheter to target implantation site.

The pacing lead conversion tool 100 can be mounted on the pacing lead 204. For example, at operation 1502, the pacing lead 204 can be inserted into the pacing lead conversion tool 100. The pacing lead 204 can be advanced proximally into the central lumen 110 until the connector pin 203 is engaged within the pin subsegment 210 of the central lumen 110 and the connector body 208 is located within the connector body subsegment 212 of the central lumen 110. The body wall 106 surrounding the central lumen 110 can engage the connector body 208 in a first friction fit and/or can engage the connector pin 203 in a second friction fit. The second friction fit can be tighter than the first friction fit.

The stylet 1102 can be inserted into the lead lumen 1104 of the pacing lead 204 to support the pacing lead 204. The stylet 1102 may be loaded through the funnel segment 220 of the central lumen 110. For example, the distal tip of the stylet 1102 can be advanced through the lead lumen 1104 such that the stylet 1102 supports the pacing lead 204 and lends stiffness to the lead assembly to assist in pushing the pacing lead 204 toward and into the target anatomy.

The stylet 1102, if being used, can be passed through the side slot 120. More particularly, rather than being loaded after the pacing lead 204 is engaged with the conversion tool, the stylet 1102 may be loaded into the pacing lead 204 and then the lead/stylet 1102 assembly can be loaded into the conversion tool. The stylet 1102 can be passed laterally through the side slot 120 between the central lumen 110 and the surrounding environment 122. The pacing lead 204 may then be retracted to locate the connector pin 203 within the pin subsegment 210.

At operation 1504, the pacing lead conversion tool 100 can be torqued such that the connector pin 203 and the pacing lead conversion tool 100 rotate relative to the connector body 208. The applied torque can be above a predetermined value such that the tool body 102 slides over the connector body 208. The tool body 102 may nonetheless remain fixed to the connector pin 203. Accordingly, the connector pin 203 can move relative to the connector body 208.

Rotation of the connector pin 203 can cause the helix to be extended. Accordingly, the helix can be extended (or retracted) by rotating the tool body 102 while stabilizing the lead body 206. The physician may then manipulate the lead body 206 to drive the distal end of the pacing lead 204 through the septal tissue to the target pacing site, e.g., the LBB. During advancement, the pacing lead conversion tool 100 can apply rotational forward pressure on the helix so that it maintains its extended state even if forces are applied to the helix. The helix can therefore remain fixed and may not spontaneously retract during implantation of the pacing lead 204.

At operation 1506, the electrical connector 1402 can be attached to the pacing lead 204. More particularly, the electrical connector 1402 can be engaged to the connector pin 203 and/or connector body 208 through the connector openings 128 of the pacing lead conversion tool 100. The electrical connectors 1402 may convey electrical signals used to confirm proper lead placement, e.g., via determination by the PSA. When proper placement is confirmed, the electrical connector 1402 and the pacing lead conversion tool 100 can be removed from the pacing lead 204. The pacing lead 204 may then be inserted into a pacing device, e.g., a pacemaker, to provide electrical stimulation to the target tissue.

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

1. A lead conversion tool, comprising:
a tool body including a body wall extending around a central lumen, wherein the central lumen extends along a longitudinal axis from a distal tool end to a proximal tool end, wherein the central lumen includes a locking lumen segment having a connector body subsegment and a pin subsegment, wherein the connector body subsegment is sized to receive a connector body of a lead in a first friction fit, and wherein the pin subsegment is sized to receive a connector pin of the lead in a second friction fit that is tighter than the first friction fit.

2. The lead conversion tool of claim 1, wherein the connector body subsegment of the locking lumen segment has a larger diameter than the pin subsegment of the locking lumen segment.

3. The lead conversion tool of claim 1, wherein the central lumen includes a funnel lumen segment proximal to the locking lumen segment.

4. The lead conversion tool of claim 1 further comprising a side slot extending through the body wall of the tool body from the central lumen to a surrounding environment.

5. The lead conversion tool of claim 4, wherein the side slot includes an intermittent slot having a plurality of slot segments longitudinally separated by a plurality of connector openings.

6. The lead conversion tool of claim 5, wherein the plurality of slot segments and the plurality of connector openings combine to form a continuous opening from the proximal tool end to the distal tool end.

7. The lead conversion tool of claim 5, wherein one or more of the plurality of connector openings includes a guide surface tapering radially inward from an outer surface of the body wall to a clip surface.

8. The lead conversion tool of claim 1 further comprising a plurality of relief slots extending longitudinally from the distal tool end through the body wall radially outward of the connector body subsegment.

9. The lead conversion tool of claim 1 further comprising a pin sleeve within the pin subsegment, wherein the pin sleeve comprises a material softer than a material of the body wall.

10. A lead conversion tool, comprising:
a tool body including a body wall extending around a central lumen, wherein the central lumen extends along a longitudinal axis from a distal tool end to a proximal tool end, wherein the central lumen includes a locking lumen segment and a funnel segment, wherein the locking lumen segment includes a connector body subsegment and a pin subsegment, wherein the funnel segment tapers from a proximal opening at the proximal tool end to a distal opening adjacent to the pin subsegment, and wherein the distal opening has a smaller diameter than the pin subsegment.

11. The lead conversion tool of claim 10, wherein the pin subsegment has a smaller diameter than the connector body subsegment.

12. The lead conversion tool of claim 10, wherein the proximal opening has a larger diameter than the connector body subsegment, the pin subsegment, and the distal opening.

13. The lead conversion tool of claim 10 further comprising a side slot extending through the body wall of the tool body from the central lumen to a surrounding environment, wherein the side slot extends from the distal tool end to the proximal tool end.

14. The lead conversion tool of claim 13, wherein the side slot extends through the funnel segment.

15. The lead conversion tool of claim 10, wherein the connector body subsegment is sized to receive a connector body of a lead in a first friction fit, and wherein the pin subsegment is sized to receive a connector pin of the lead in a second friction fit that is tighter than the first friction fit.

16. The lead conversion tool of claim 10 further comprising a pin sleeve within the pin subsegment, wherein the pin sleeve comprises a material softer than a material of the body wall.

17. A method, comprising:
inserting a lead into a lead conversion tool such that a connector body of the lead is received in a central lumen of the lead conversion tool in a first friction fit and a connector pin of the lead is received within the central lumen of the lead conversion tool in a second friction fit that is tighter than the first friction fit; and
torquing the lead conversion tool such that the connector pin and the lead conversion tool rotate relative to the connector body.

18. The method of claim 17 further comprising inserting a stylet into a lead lumen of the lead through a funnel segment of the central lumen, wherein the funnel segment tapers from a proximal opening to a distal opening adjacent to a pin subsegment of the central lumen, and wherein the distal opening has a smaller diameter than the pin subsegment.

19. The method of claim 17 further comprising passing a stylet through a side slot extending through a body wall of the lead conversion tool, wherein the stylet passes laterally through the side slot between the central lumen and a surrounding environment.

20. The method of claim 17 further comprising engaging an electrical connector to the connector pin within a pin subsegment of the central lumen through a connector opening extending through a body wall of the lead conversion tool.
